(19) United States
(12) Patent Application Publication
(10) Pub. No.: US 2025/0067718 A1
(43) Pub. Date: Feb. 27, 2025

(54) METHOD FOR EVALUATING AND RANKING THE ENHANCED RECOVERY POTENTIAL FOR UNCONVENTIONAL RESERVOIRS

(71) Applicant: ARAMCO SERVICES COMPANY, Houston, TX (US)

(72) Inventors: Anuj Gupta, Katy, TX (US); Jewel Duncan, Houston, TX (US); Stacey M. Althaus, Houston, TX (US); Shannon Lee Eichmann, Katy, TX (US)

(73) Assignee: ARAMCO SERVICES COMPANY, Houston, TX (US)

(21) Appl. No.: 18/456,438

(22) Filed: Aug. 25, 2023

(51) Int. Cl. G01N 33/24; E21B 41/00; E21B 43/14; E21B 43/16; E21B 49/02; G01N 24/08

(52) U.S. Cl. CPC G01N 33/241; E21B 41/0064; E21B 43/14; E21B 43/16; E21B 49/02; G01N 24/081

(57) ABSTRACT

A method to perform a field operation of an unconventional reservoir is disclosed. The method includes selecting, from rock samples and based on test reports of corresponding companion rock samples, selected rock samples representing rock characteristics of formation zones in the unconventional reservoir, performing, using a rock sample test apparatus, a sequence of rock sample test cycles on each selected rock sample to generate rock sample test results, generating, based on the rock sample test results, a ranking of the selected rock samples representing enhanced oil recovery (EOR) potential of the formation zones, selecting, from the formation zones and based on the ranking, a target formation zone having an EOR potential meeting a pre-determined criterion, and performing, based at least on the EOR potential of the target formation zone, the field production of the unconventional reservoir.

### BACKGROUND

An unconventional reservoir has hydrocarbons formed within the rock and never migrated, such as shale, coal, tight-sand, and oil-sand. These reservoirs contain enormous quantities of oil and natural gas but are challenging to produce economically on a commercial scale. In contrast, the conventional reservoir is a porous and permeable rock formation where hydrocarbons have migrated from a source rock. Unconventional reservoirs around the world are characterized by very low depletion based primary recovery such that a large potential remains for enhancing the recovery.

In the oil and gas industry, the term “Huff and Puff” (HnP) refers to a cyclic process in which a well is injected with a recovery enhancement fluid and, after a soak period, the well is put back on production. The HnP process is one of the enhanced oil recovery (EOR) processes, which is the practice of extracting oil from a well that has already gone through the primary depletion stage of oil recovery.

### SUMMARY

In general, in one aspect, the invention relates to a method to perform a field operation of an unconventional reservoir. The method includes selecting, from a plurality of rock samples and based on test reports of corresponding companion rock samples, a plurality of selected rock samples representing rock characteristics of a plurality of formation zones in the unconventional reservoir, performing, using a rock sample test apparatus, a sequence of rock sample test cycles on each selected rock sample of the plurality of selected rock samples to generate rock sample test results, generating, based on the rock sample test results, a ranking of the plurality of selected rock samples representing enhanced oil recovery (EOR) potential of the plurality of formation zones, selecting, from the plurality of formation zones and based on the ranking of the plurality of selected rock samples, a first target formation zone having a first EOR potential meeting a first pre-determined criterion, and performing, based at least on the first EOR potential of the first target formation zone, the field production of the unconventional reservoir.

In general, in one aspect, the invention relates to a core sample analysis system to facilitate a field operation of an unconventional reservoir. The core sample analysis system includes a computer processor and memory storing instructions, when executed by the computer processor comprising functionality for selecting, from a plurality of rock samples and based on test reports of corresponding companion rock samples, a plurality of selected rock samples representing rock characteristics of a plurality of formation zones in the unconventional reservoir, performing, using a rock sample test apparatus, a sequence of rock sample test cycles on each selected rock sample of the plurality of selected rock samples to generate rock sample test results, generating, based on the rock sample test results, a ranking of the plurality of selected rock samples representing enhanced oil recovery (EOR) potential of the plurality of formation zones, selecting, from the plurality of formation zones and based on the ranking of the plurality of selected rock samples, a first target formation zone having a first EOR potential meeting a first pre-determined criterion, and facilitating, based at least on the first EOR potential of the first target formation zone, the field production of the unconventional reservoir.

In general, in one aspect, the invention relates to a system that includes a well control system for performing a field operation of an unconventional reservoir and a core sample analysis system having a computer processor and memory storing instructions. The instructions when executed by the computer processor comprising functionality for selecting, from a plurality of rock samples and based on test reports of corresponding companion rock samples, a plurality of selected rock samples representing rock characteristics of a plurality of formation zones in the unconventional reservoir, performing, using a rock sample test apparatus, a sequence of rock sample test cycles on each selected rock sample of the plurality of selected rock samples to generate rock sample test results, generating, based on the rock sample test results, a ranking of the plurality of selected rock samples representing enhanced oil recovery (EOR) potential of the plurality of formation zones, selecting, from the plurality of formation zones and based on the ranking of the plurality of selected rock samples, a first target formation zone having a first EOR potential meeting a first pre-determined criterion, and facilitating, based at least on the first EOR potential of the first target formation zone, the well control system to perform the field production of the unconventional reservoir.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 shows a system in accordance with one or more embodiments.

FIG. 2 shows a method flowchart in accordance with one or more embodiments.

FIGS. 3A-3M show example apparatuses and graphs in accordance with one or more embodiments.

FIG. 4 shows a computing system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (for example, first, second, third) may be used as an adjective for an element (that is, any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure include a method and system for quantifying the amount of oil that is left behind in different geological formation zones of an unconventional reservoir after complete primary depletion. Further, embodiments disclosed herein involve measuring the percentage of residual oil that may be recoverable from such an unconventional reservoir. In particular, the residual oil is to be recovered using a carbon dioxide (CO₂) Huff-n-Puff enhanced recovery (HnP EOR) method. Further, the amount of CO₂ that may be stored in the reservoir is estimated if CO₂ is injected (or reinjected) at the end of the enhanced oil recovery operations.

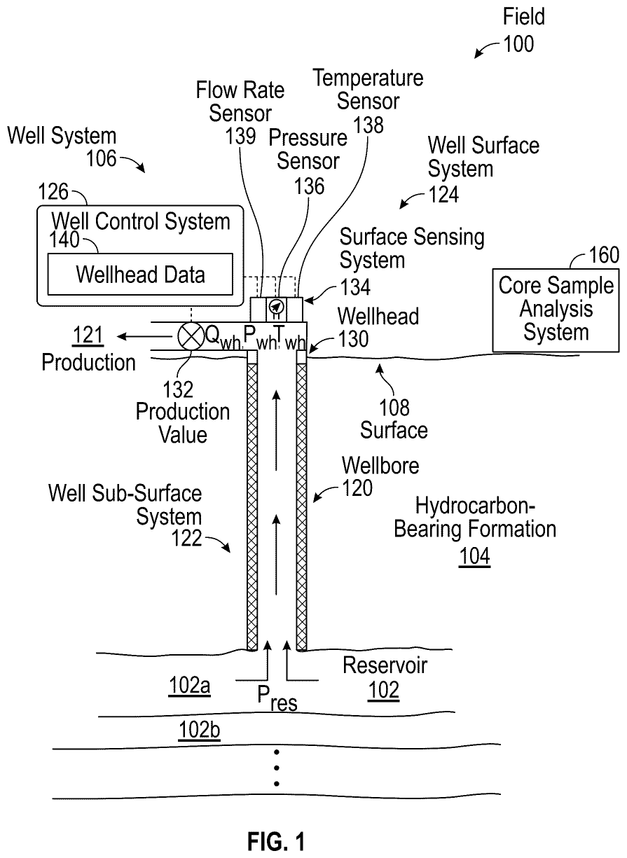

FIG. 1 shows a schematic diagram in accordance with one or more embodiments. More specifically, FIG. 1 illustrates a field (100) that includes a hydrocarbon reservoir (“reservoir”) (102) located in a subsurface hydrocarbon-bearing formation (“formation”) (104) and a well system (106). The hydrocarbon-bearing formation (104) may include a porous or fractured rock formation that resides underground, beneath the Earth's surface (“surface”) (108). In the case of the well system (106) being a hydrocarbon well, the reservoir (102) may include a portion of the hydrocarbon-bearing formation (104). In one or more embodiments, the reservoir (102) is an unconventional reservoir. The hydrocarbon-bearing formation (104) and the reservoir (102) may include different layers of rock (referred to as formation layers, such as formation layers (102a, 102b, etc.)) having varying characteristics, such as varying degrees of permeability, porosity, density, capillary pressure, acoustic, radioactive properties and resistivity. In the case of the well system (106) being operated as a production well, the well system (106) may facilitate the extraction of hydrocarbons (or “production”) from the reservoir (102).

In some embodiments, the well system (106) includes a wellbore (120), a well sub-surface system (122), a well surface system (124), a well control system (“control system”) (126) and a core sample analysis system (160). The wellbore (120) may include a bored hole that extends from the surface (108) into a target zone of the hydrocarbon-bearing formation (104), such as the reservoir (102). An upper end of the wellbore (120), terminating at or near the surface (108), may be referred to as the “up-hole” end of the wellbore (120), and a lower end of the wellbore, terminating in the hydrocarbon-bearing formation (104), may be referred to as the “down-hole” end of the wellbore (120). The wellbore (120) may facilitate the circulation of drilling fluids during drilling operations, the flow of hydrocarbon production (“production”) (121) (e.g., oil and gas) from the reservoir (102) to the surface (108) during production operations, the injection of substances (e.g., gas, water) into the hydrocarbon-bearing formation (104) or the reservoir (102) during injection operations, or the communication of monitoring devices (e.g., logging tools) into the hydrocarbon-bearing formation (104) or the reservoir (102) during monitoring operations (e.g., during in situ logging operations).

In some embodiments, the well sub-surface system (122) includes casing installed in the wellbore (120). For example, the wellbore (120) may have a cased portion and an uncased (or “open-hole”) portion. The cased portion may include a portion of the wellbore having casing (e.g., casing pipe and casing cement) disposed therein. The uncased portion may include a portion of the wellbore not having casing disposed therein. In some embodiments, the casing includes an annular casing that lines the wall of the wellbore (120) to define a central passage that provides a conduit for the transport of tools and substances through the wellbore (120). For example, the central passage may provide a conduit for lowering logging tools into the wellbore (120), a conduit for the flow of production (121) (e.g., oil and gas) from the reservoir (102) to the surface (108), or a conduit for the flow of injection substances (e.g., gas, water) from the surface (108) into the hydrocarbon-bearing formation (104). In some embodiments, the well sub-surface system (122) includes production tubing installed in the wellbore (120). The production tubing may provide a conduit for the transport of tools and substances through the wellbore (120). The production tubing may, for example, be installed inside casing. In such an embodiment, the production tubing may provide a conduit for some or all of the production (121) (e.g., oil and gas, water) passing through the wellbore (120) and the casing.

In some embodiments, the well surface system (124) includes a wellhead (130). The wellhead (130) may include a rigid structure installed at the “up-hole” end of the wellbore (120), at or near where the wellbore (120) terminates at the Earth's surface (108). The wellhead (130) may include structures for supporting (or “hanging”) casing and production tubing extending into the wellbore (120). Production (121) may flow through the wellhead (130), after exiting the wellbore (120) and the well sub-surface system (122), including, for example, the casing and the production tubing. In some embodiments, the well surface system (124) includes flow regulating devices that are operable to control the flow of substances into and out of the wellbore (120). For example, the well surface system (124) may include one or more production valves (132) that are operable to control the flow of production (121). For example, a production valve (132) may be fully opened to enable unrestricted flow of production (121) from the wellbore (120), the production valve (132) may be partially opened to partially restrict (or “throttle”) the flow of production (121) from the wellbore (120), and production valve (132) may be fully closed to fully restrict (or “block”) the flow of production (121) from the wellbore (120), and through the well surface system (124).

Keeping with FIG. 1, in some embodiments, the well surface system (124) includes a surface sensing system (134). The surface sensing system (134) may include sensors for sensing characteristics of substances, including production (121), passing through or otherwise located in the well surface system (124). The characteristics may include, for example, pressure, temperature, and flow rate of production (121) flowing through the wellhead (130), or other conduits of the well surface system (124), after exiting the wellbore (120).

In some embodiments, the surface sensing system (134) includes a surface pressure sensor (136) operable to sense the pressure of production (121) flowing through the well surface system (124), after it exits the wellbore (120). The surface pressure sensor (136) may include, for example, a wellhead pressure sensor that senses a pressure of production (121) flowing through or otherwise located in the wellhead (130). In some embodiments, the surface sensing system (134) includes a surface temperature sensor (138) operable to sense the temperature of production (121) flowing through the well surface system (124), after it exits the wellbore (120). The surface temperature sensor (138) may include, for example, a wellhead temperature sensor that senses a temperature of production (121) flowing through or otherwise located in the wellhead (130), referred to as “wellhead temperature” (T_{wh}). In some embodiments, the surface sensing system (134) includes a flow rate sensor (139) operable to sense the flow rate of production (121) flowing through the well surface system (124), after it exits the wellbore (120). The flow rate sensor (139) may include hardware that senses a flow rate of production (121) (Q_{wh}) passing through the wellhead (130).

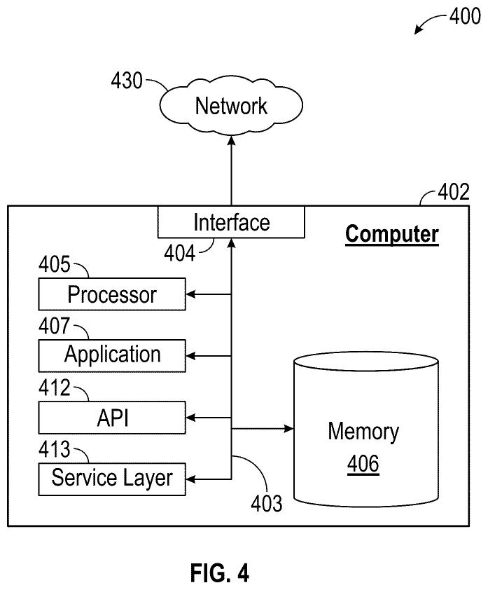

The control system (126) may control various field operations of the well system (106), such as well production operations, well injection operations, well completion operations, well maintenance operations, subsurface gas storage operations, and reservoir monitoring, assessment and development operations. In some embodiments, during operation of the well system (106), the control system (126) collects and records wellhead data (140) for the well system (106). The wellhead data (140) may include, for example, a record of measurements of wellhead pressure (P_{wh}) (e.g., including flowing wellhead pressure), wellhead temperature (T_{wh}) (e.g., including flowing wellhead temperature), wellhead production rate (Q_{wh}) over some or all of the life of the well system (106), and water cut data. In some embodiments, the measurements are recorded in real-time, and are available for review or use within seconds, minutes or hours of the condition being sensed (e.g., the measurements are available within 1 hour of the condition being sensed). In such an embodiment, the wellhead data (140) may be referred to as “real-time” wellhead data (140). Real-time wellhead data (140) may enable an operator of the well system (106) to assess a current state of the well system (106) and make real-time decisions regarding development of the well system (106) and the reservoir (102), such as on-demand adjustments in regulation of production flow from the well. In some embodiments, the control system (126) includes a computer system that is similar to the computer system (400) described below with regard to FIG. 4 and the accompanying description.

In one or more embodiments of the disclosure, the reservoir (102) is an unconventional reservoir that is highly laminated with minimal communication between different stacked geological formation layers (e.g., layers (102a, 102b, etc.) even when they are adjacent to each other vertically. Some of these formation layers have higher oil/gas content and better ability to produce fluids compared to other formation layers in the reservoir. As a result, these formation layers deplete to different levels and oil remaining after primary production may be different in each formation layer. Further, oil remaining after primary production may also be different for different areas within a single formation layer. Throughout this disclosure, a particular area within a particular formation layer is referred to as a formation zone. A rock core is a cylindrical section of a natural substance, such as sediment or rock. Rock cores are usually obtained by drilling with a coring bit (e.g., hollow steel tube) into the sediment or rocks. In some embodiments, rock cores at different depths are extracted through the wellbore (130) from various formation layers. In addition, multiple sets of rock cores may be extracted through multiple boreholes throughout the field (100) and correspond to a wide range of formation zones. Each rock core column is a sequence of rock cores extracted from a particular borehole and extending across a depth range of interest in the borehole. In particular, each rock core is marked to indicate the depths where it is extracted in the borehole. A core-plug is a cylindrical rock sample taken (e.g., cut or drilled) from a rock core for analysis. Core plugs are typically 1″ to 1.5″ (i.e., approximately 2.5 to 3.8 cm) in diameter and 1″ to 2″ (i.e., approximately 2.5 to 5 cm) in length. Each core-plug corresponds to a particular formation zone according to the depth markings of the rock core in the rock column. Throughout this disclosure, the term “sample” refers to a rock sample or core-plug taken from a rock core and the term “sample depth” refers to the depth where the rock sample or core-plug is extracted from the rock core.

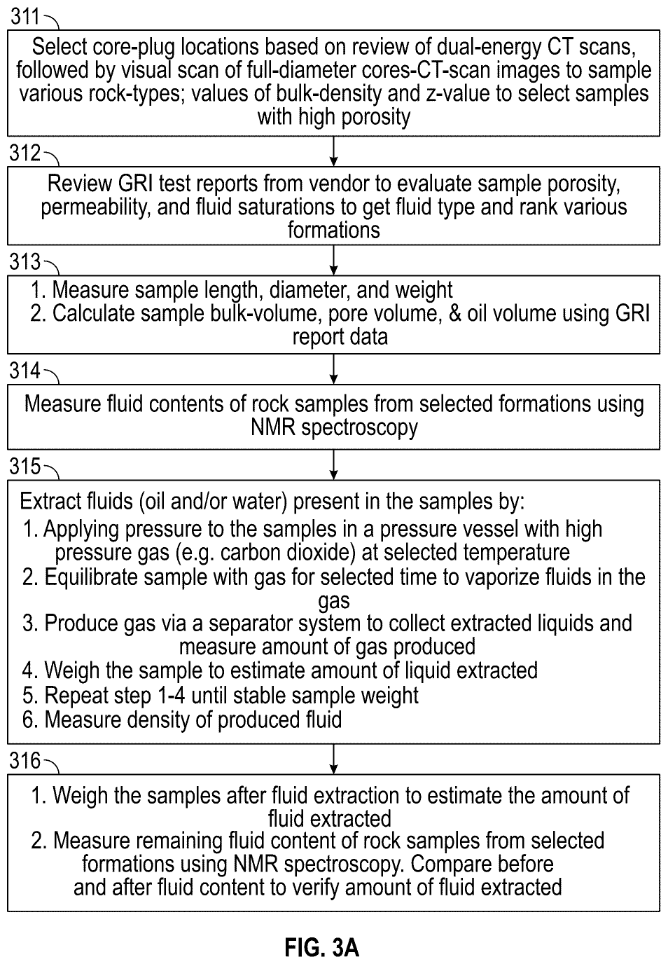

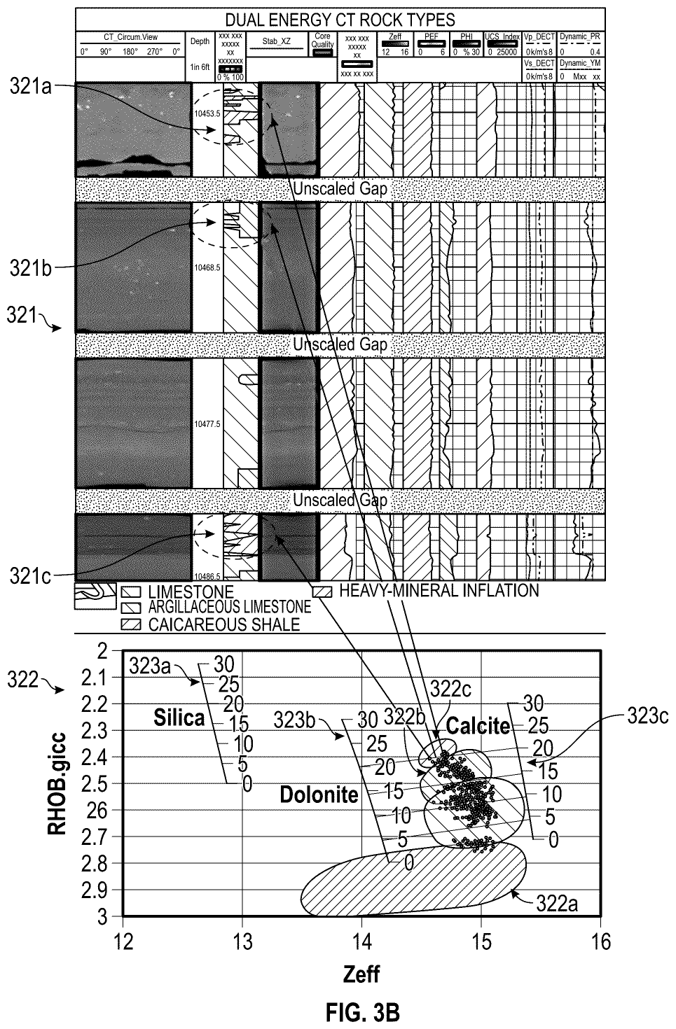

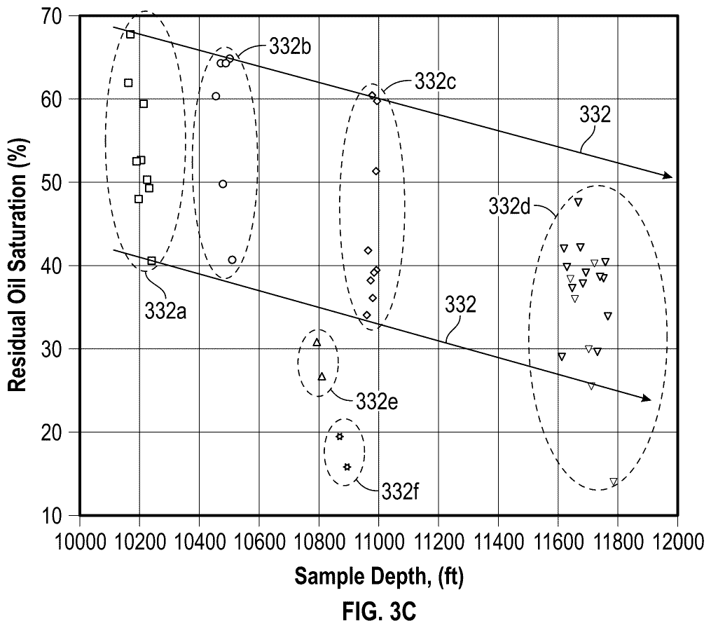

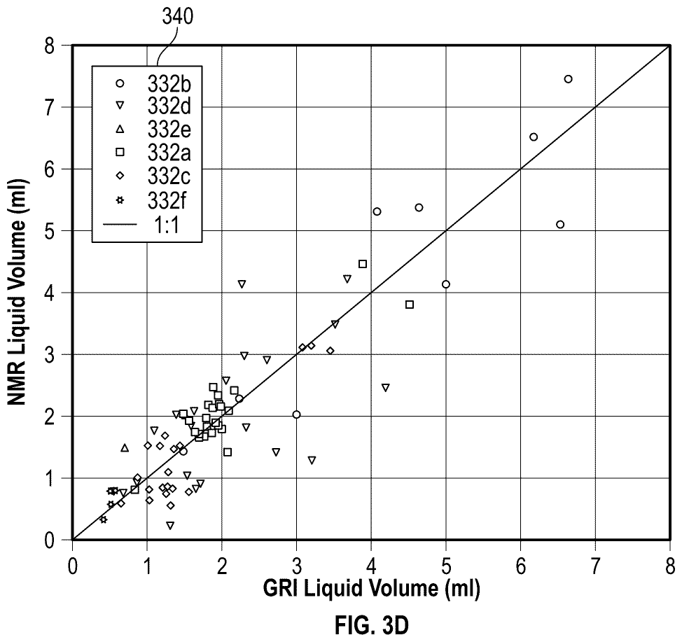

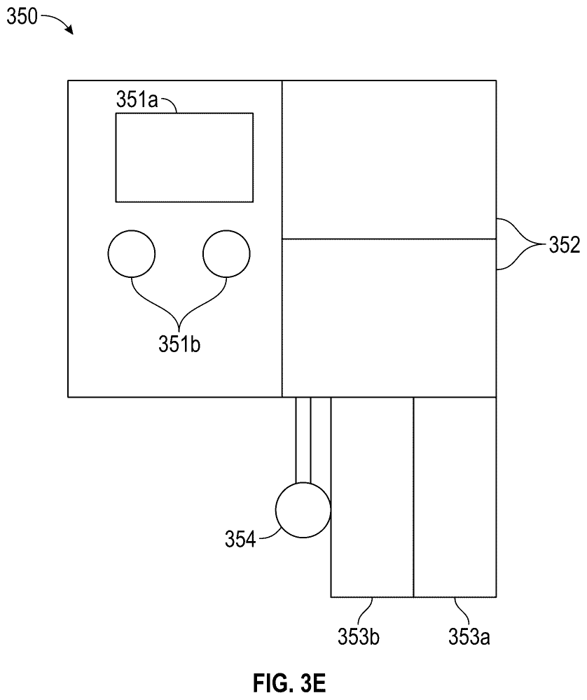

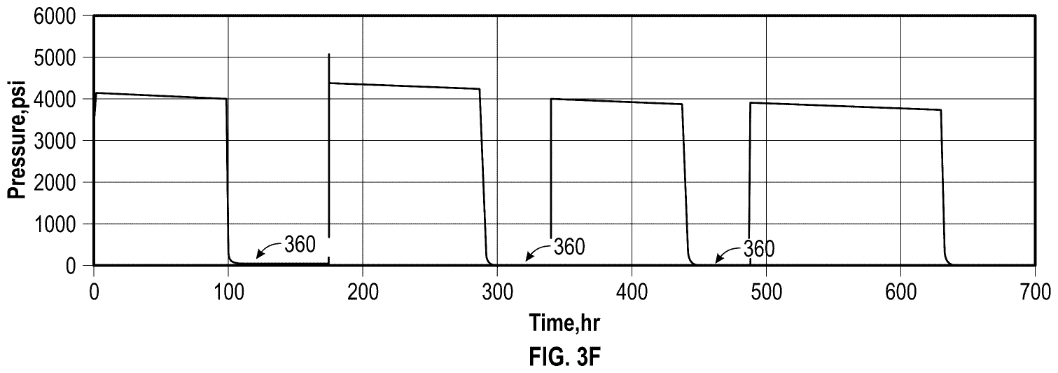

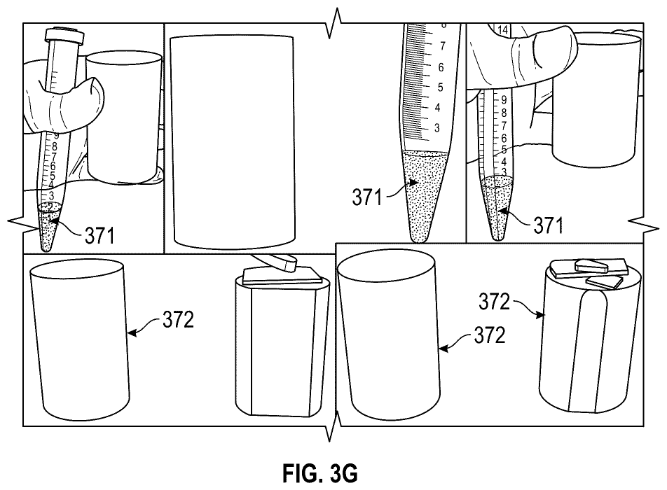

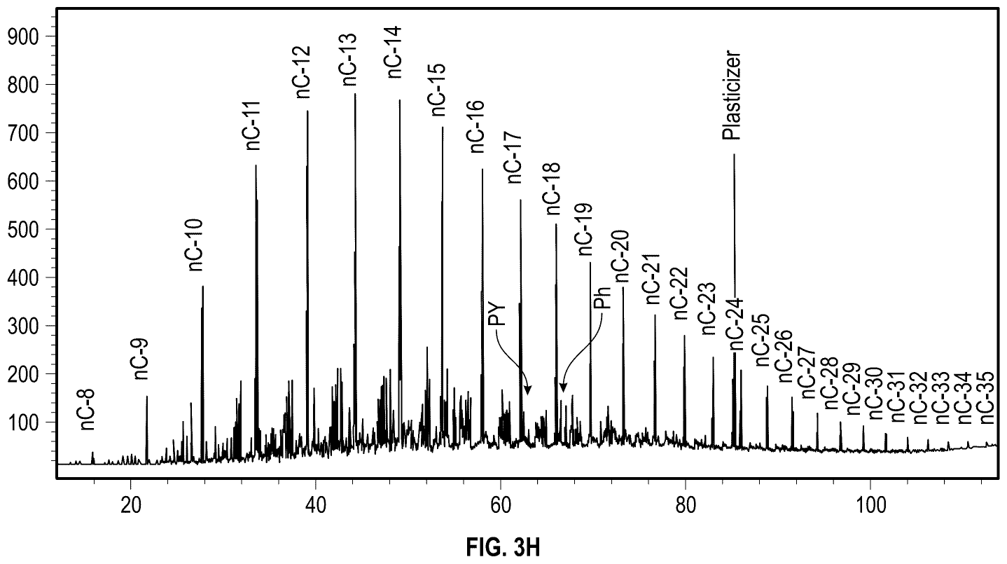

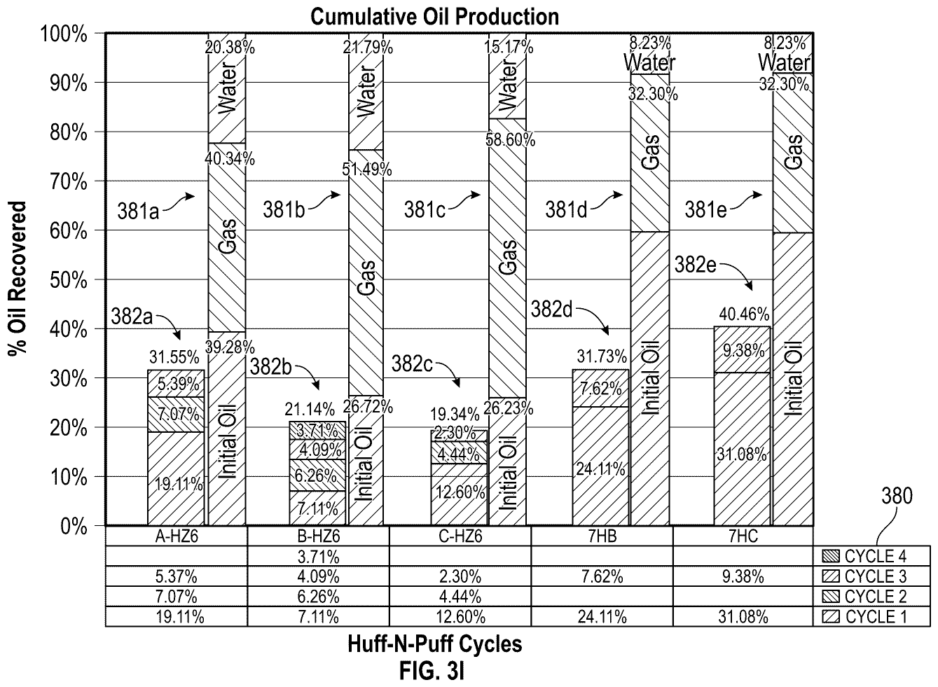

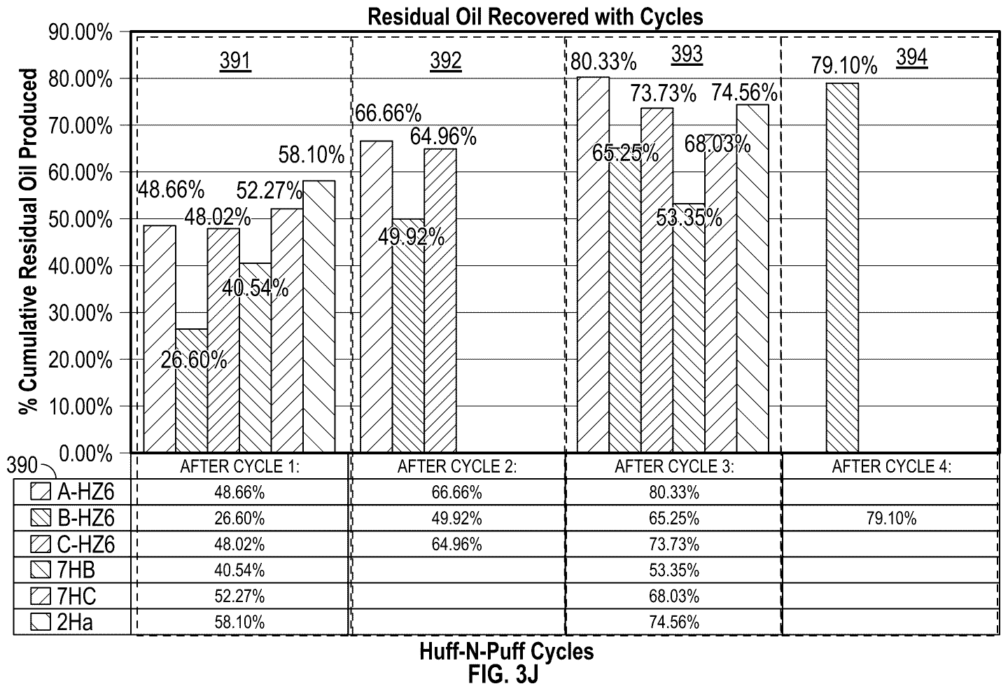

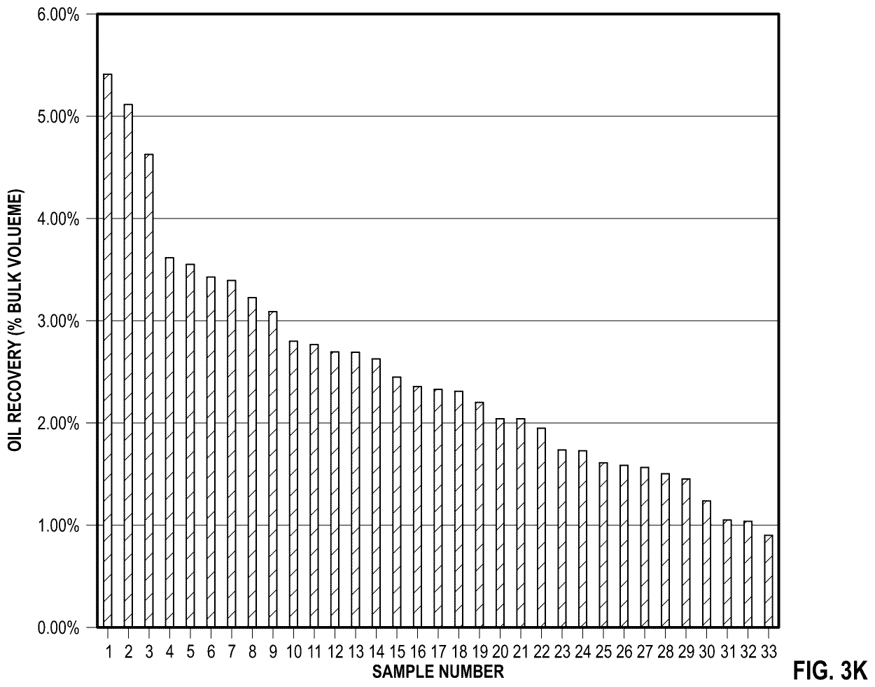

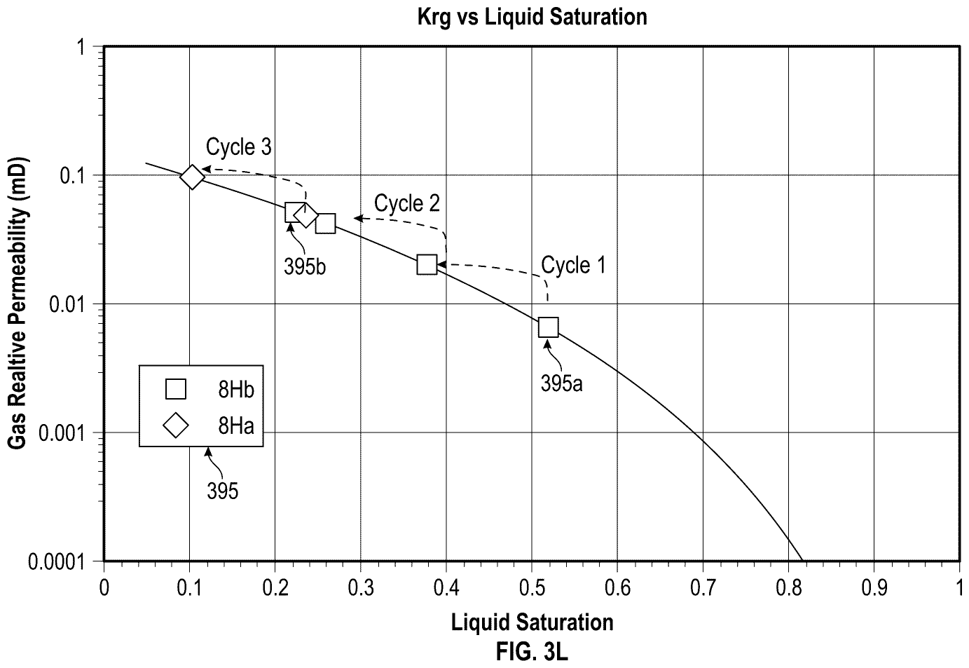

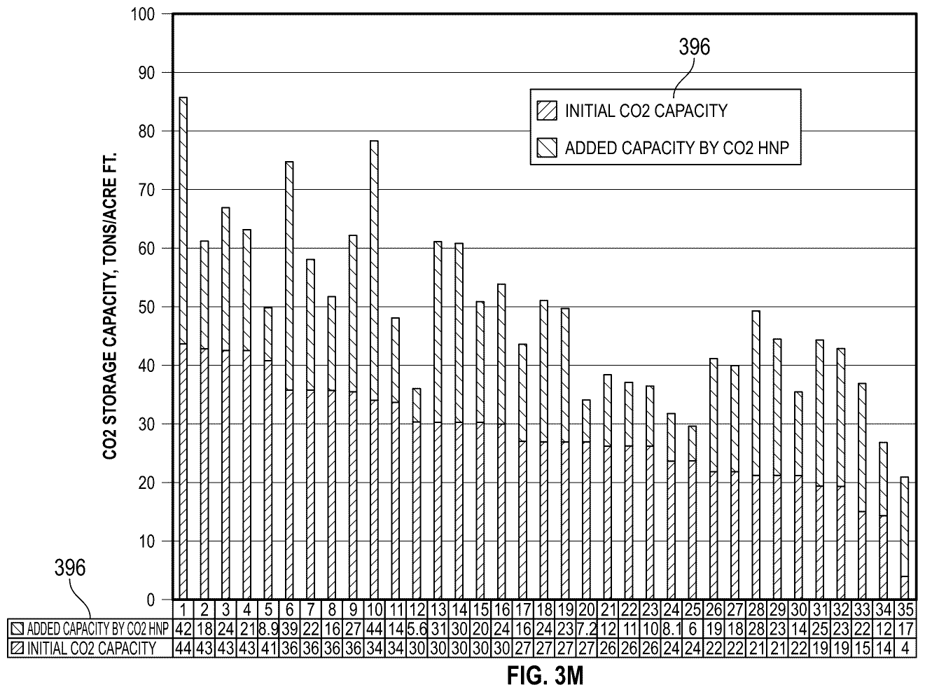

In some embodiments, the core sample analysis system (160) may include hardware and/or software with functionality for ranking core samples from various formation zones and/or determining residual oil amount and sequestration capacity of formation zones. For example, the core sample analysis system (160) may store logs and data regarding core samples for performing ranking and analysis. While the core sample analysis system (160) is shown at a well site, embodiments are contemplated where reservoir simulators are located away from well sites. In some embodiments, the core sample analysis system (160) may include a core sample test apparatus that is similar to that described below with regard to FIG. 3E and the accompanying description. Further, the core sample analysis system (160) may include a computer system that is similar to the computer system (400) described below with regard to FIG. 4 and the accompanying description.

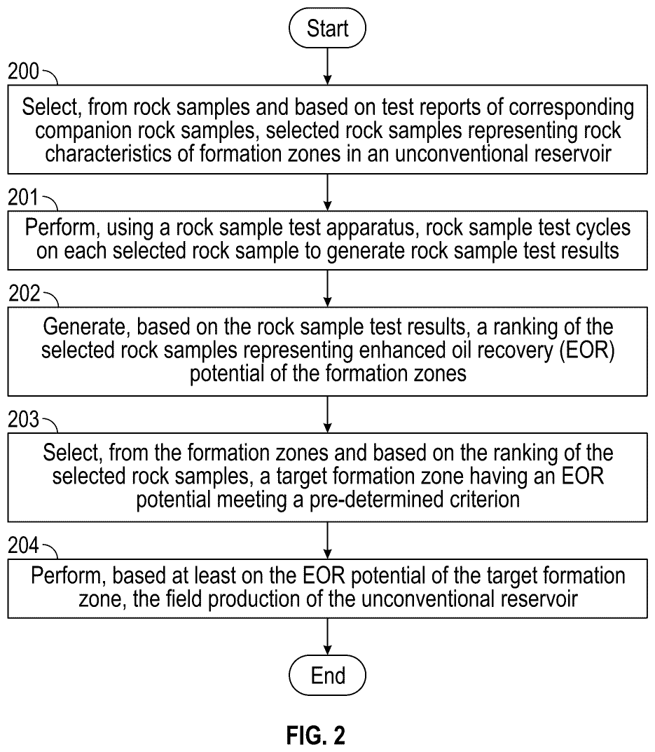

FIG. 2 shows a flowchart in accordance with one or more embodiments disclosed herein. Specifically, FIG. 2 shows a workflow for evaluating and ranking the enhanced recovery potential for unconventional reservoirs subsequent to primary recovery production. In one or more embodiments, the workflow determines a measure of incremental oil recovery of a depleted unconventional reservoir based on enhanced oil recovery methods. The measure of incremental oil recovery quantifies the incremental oil recovery potential and is used for evaluating the lifetime economic value of the unconventional reservoir and generating an effective field development plan including design of surface facilities. One or more of the steps in FIG. 2 may be performed using the components of the field (100), discussed above in reference to FIG. 1. In one or more embodiments, one or more of the steps shown in FIG. 2 may be omitted, repeated, and/or performed in a different order than the order shown in FIG. 2. Accordingly, the scope of the disclosure should not be considered limited to the specific arrangement of steps shown in FIG. 2.

Initially in Step 200, rock samples are selected based on test reports of companion rock samples. The rock samples and companion rock samples are taken from rock cores extracted from various wellbores penetrating formation layers throughout a wide range of locations in the unconventional reservoir. A particular rock sample and its companion sample are taken from nearby locations in proximity to each other on the same rock core. In particular, the rock samples are collected from various formation zones subsequent to primary depletion (i.e., oil recovery production) of the unconventional reservoir. Each selected rock sample represents rock characteristics of a formation zone where the rock sample is collected in the unconventional reservoir.

In Block 201, a sequence of rock sample test cycles using a rock sample test apparatus are performed on each of the selected rock samples to generate rock sample test results. In some embodiments, the rock sample test apparatus includes a Huff-and-Puff (HnP) test apparatus to perform a sequence of HnP cycles on each rock sample. Throughout this disclosure, the term “HnP cycle” refers to a laboratory HnP cycle unless explicitly specified otherwise. The laboratory HnP cycle is a process performed in a laboratory at the Earth's surface where a rock sample is placed inside a pressure vessel filled with a particular gas, such as carbon dioxide (CO₂). The rock sample remains in the pressurized gas for a time period referred to as a soaking period. The pressure vessel is brought back to the atmospheric condition (i.e., one atmosphere (ATM)) subsequent to the soaking period when residual fluids produced from the rock sample are collected for analysis. This completes one HnP cycle. In some embodiments, each selected rock sample is measured prior to the sequence of rock sample test cycles (e.g., 2, 3, 4 cycles, etc.) to generate prior weight and volume measurements and NMR measurements. In addition, each selected rock sample is also measured after the sequence of rock sample test cycles to generate subsequent weight and volume measurements and NMR measurements. Accordingly, the prior weight and volume measurements and the subsequent weight and volume measurements are compared to determine a residual oil amount of each selected rock sample based on the weight difference and volume difference. For example, the weight difference corresponds to the weight of the oil recovered (i.e., separated and collected) from the selected rock sample by way of the HnP cycles. In addition, the prior NMR measurements and subsequent NMR measurements are analyzed to determine the type and amount of hydrocarbons contained in the selected rock sample before and after the HnP cycles. The rock sample test results thus include the amount and type of residual hydrocarbons in each selected rock sample before and after the HnP cycles.

In Block 202, a ranking of the selected rock samples is generated based on the rock sample test results to represent enhanced oil recovery (EOR) potential of the corresponding formation zones. In other words, the sample having a higher ranking indicates that the formation zone where the sample is collected has higher EOR potential.

In Block 203, one or more target formation zones are selected from all the formation zones based on the ranking of the selected rock samples. In some embodiments, the selected target formation zone is further based on its EOR potential meeting a pre-determined criterion, such as exceeding a minimum residual oil percentage. In some embodiments, a storage capacity for carbon dioxide (CO₂) sequestration of the target formation zone is determined based on the rock sample test results.

In Block 204, a field production of the unconventional reservoir is performed based at least on the EOR potential of the target formation zone. In some embodiments, the field operation includes an EOR production operation to extract hydrocarbons from the target formation zone. In some embodiments, the field operation includes a sequestration operation to store carbon dioxide (CO₂) gas in the target formation zone subsequent to the EOR production operation. For example, the gas stored in a particular target zone may be used to perform a separate EOR production operation of another selected target zone that is within a pre-determined range (e.g., 1 mile) from the particular target zone. In other words, the separate EOR production operation includes an injection operation using the stored gas.

FIGS. 3A-3M show an implementation example in accordance with one or more embodiments. Specifically, FIGS. 3A-3M collectively illustrate a work flow for quantifying the amount of residual oil with recoverable percentage and estimating sequestration capacity in different geological formation zones of an unconventional reservoir after complete primary depletion. The implementation example shown in FIGS. 3A-3M is based on the system and method flowchart described in reference to FIGS. 1 and 2 above.

FIG. 3A shows a process flow diagram of ranking various geological formation zones. Initially in Block 311, core-plug locations for cutting/drilling from the core column are selected based on review of dual-energy CT scans followed by a visual scan of the full-diameter core column. Dual-energy CT scans refer to scans on a core column at the surface. These scans provide a very high resolution measure (0.25-1.0 mm resolution) of the core material bulk-density and average atomic number (or Photoelectric Index). Some well-logs measured in the borehole (e.g. lithodensity logs) obtain measurements similar measurements of bulk-density and photoelectric index but at much lower resolution (1-3 ft). These two measurements, when used carefully allow calculation of the porosity of the core column.

Each selected core-plug location corresponds to a formation zone according to depth markings on the rock cores of the core column. An example of the dual-energy CT scans and selection criteria are described in reference to FIG. 3B below. The visual scan of the full-diameter allows identification of intervals that are rich in organic matter and thereby potentially rich in hydrocarbon filled pore-space. The visual scans may also allow identification of the extent of laminations and occurrence of fractures. Such features may enhance the flow capacity but also may make it more difficult to measure the flow and EOR potential of the samples.

In Block 312, rock sample characteristics (e.g., porosity, permeability, fluid saturations) of sample cores (e.g., core-plugs) from selected locations (i.e., formation zones) are evaluated based on test reports to determine fluid types and to rank various formation zones. GRI analysis reports include measurements of sample bulk-density, grain density, porosity, permeability and saturation of oil, water and gas. Such properties allow calculation of the flow and storage capacity of different sample as well as the residual oil and gas saturation in the samples. These characteristics are used to rank the samples.

In one or more embodiments, the test reports include GRI test reports provided by a third party testing vendor. For example, the GRI test report of a particular rock sample may be performed on a companion sample previously sent to the third party testing vendor that was extracted from the same formation zone (i.e., same depth in the same borehole) as the particular rock sample. As described above, the particular rock sample and the companion sample are taken from nearby locations in proximity to each other on the same rock core. An example of reservoir fluid identification and core sample selection are described in reference to FIG. 3C below.

In Block 313, each sample is measured for length, diameter, and weight. The bulk volume, pore volume, and oil volume of each sample are calculated based on the GRI reports.

In Block 314, the fluid content of the selected samples are measured using Nuclear Magnetic Resonance (NMR) spectroscopy. An example of measuring fluid-contents of the selected samples is described in reference to FIG. 3D below.

In Block 315, fluids (e.g., oil and/or water) are extracted from the selected sample after loading into a pressure vessel. After loading, the pressure vessel is pressured up with CO₂ (or other gases such as methane, ethane, propane or a mixture of gases) above or below the expected minimum miscibility pressure (MMP) for a specified time duration ranging from a few hours to several days to allow the fluids in the core sample to vaporize in the surrounding gas. Some of the gas may also dissolve in the oil present in the pore-space causing the oil volume to increase, referred to as swelling. After the specified time duration when the pressure vessel returns to atmospheric pressure, the gas mixture surrounding the core sample, with additional fluid vaporized from the core sample, is then produced from the pressure vessel by gradually reducing the pressure and via a separator. The separator is used to collect and measure the amount of condensed liquids and measure the amount of produced gas. The process is repeated until no additional liquid is produced from the pressure vessel and the sample weight stabilizes. The produced fluid is analyzed to determine and measure the quantity, density and composition.

In Block 316, the final sample weight is measured and compared to the sample weight measured in Block 313 to determine the amount of extracted fluids. The post-testing NMR evaluation is performed to determine the remaining fluid content of the rock sample, which is compared to the initial fluid content measurements obtained in Block 314 to determine the amount of hydrocarbons extracted from the sample. Weight and NMR measurement can provide the estimates of recovery on their own. However, since each measurement has inherent uncertainty using both methods gives a more reliable measurement of recovery. In general, weight measurement is faster and less expensive and can provide a first order working estimate.

FIG. 3B illustrates selecting core-plug locations using dual-energy CT scan results. As noted above, the locations for drilling core plugs were selected based on reviewing dual-energy CT (DECT) scans which provides accurate density and effective atomic number (Z_{eff}) of formation zones. As shown in FIG. 3B, the data tracks (321) show DECT scan results for a section of Well A that include the measured DECT density, DECT Z_{eff}, and inferred photoelectric effect (PE), porosity, unconfined compressive strength (UCS), compressional acoustic velocity (Vp), Shear acoustic velocity (Vs), dynamic Poisson's ratio, and Young's modulus. In addition, the cross-plot (322) shows the DECT bulk density versus DECT Z_{eff} from well A. For example, the depth ranges (321a), (321b), and (321c) in the data tracks (321) correspond to the data point clusters (322a), (322b), and (322c), respectively in the cross-plot (322). Further, the cross-plot (322) is superimposed by vertically running estimated porosity scale bars (322a, 322b, 322c) pure inorganic minerals (i.e., Silica, Dolomite, and Calcite) without organic matter content. Porosity values indicate the fraction of rock volume available for the storage of oil, gas, water or any injected gas such as CO₂. A high value of porosity indicates that a given volume of rock can store larger amounts of fluid filling up its pore space. Porosity scale bars of these plots can be used to estimate the porosity of the sample based on where its DECT density and DECT Z_{eff} plot. The porosity estimates in the estimated porosity scale bars (322a, 322b, 322c) can be interpreted erroneously because increasing organic content can reduce both density and Z_{eff}. For example, even though the porosity values indicated by the dolomite estimated porosity scale bar (323b) in the data cluster (322a) for rock type at depth A in the depth range (321a) is close to zero on the dolomite estimated porosity scale bar (323b), the core sample collected at that depth is reported to have a porosity of 8.5% based on GRI analysis. The sample location selection is guided based on DECT scan results to choose core-plug locations in the whole core column where there is limited fracturing and covers a diversity of rock types. Similar DECT scan results for other wells are used to guide the selection of core-plug locations in the core column from other wells.

The core-plugs taken (e.g., drilled) from preserved rock cores are designated “as-received core-plug” samples. As discussed above in Block 311, such as-received core-plug samples were initially screened based on the GRI test results conducted on companion cores samples. A small portion of data in the GRI report is shown in Table 1.

TABLE 1
Preliminary Ranking of Various Samples Based on GRI Analysis

| | | Matrix | Bulk | Grain | | | | |
|---|---|---|---|---|---|---|---|---|
| | Depth | Permeability - | Density - | Density - | Porosity - | Gas | Oil | Water |
| Well | (ft) | mD | g/cm3 | g/cm3 | Total | Saturation | Saturation | Saturation |
| A | X158 | 1.08E−04 | 2.492 | 2.6 | 5.89 | 8.73 | 61.94 | 29.33 |
| A | X167 | 2.26E−04 | 2.349 | 2.49 | 7.7 | 24.03 | 67.88 | 8.09 |
| A | X185 | 6.18E−04 | 2.307 | 2.49 | 9.72 | 35.55 | 52.51 | 11.94 |
| A | X194 | 3.98E−04 | 2.36 | 2.56 | 9.5 | 47.87 | 48 | 4.13 |
| B | X453 | 2.15E−04 | 2.49 | 2.66 | 8.54 | 22.68 | 60.37 | 16.95 |
| B | X469 | 2.59E−04 | 2.46 | 2.63 | 8.85 | 28.19 | 64.33 | 7.48 |
| B | X477 | 5.62E−04 | 2.41 | 2.63 | 10.28 | 44.35 | 49.79 | 5.86 |
| B | X486 | 9.23E−04 | 2.25 | 2.49 | 12.99 | 29.98 | 64.26 | 5.76 |
| B | X498 | 2.11E−03 | 2.25 | 2.54 | 14.9 | 30.81 | 64.83 | 4.36 |
| C | X619 | 3.07E−04 | 2.41 | 2.6 | 8.79 | 52.16 | 42.11 | 5.73 |
| C | X646 | 9.78E−04 | 2.275 | 2.57 | 13.21 | 58.4 | 37.45 | 4.15 |
| C | X655 | 7.69E−04 | 2.318 | 2.6 | 12.63 | 58.5 | 36.07 | 5.43 |
| C | X664 | 4.14E−04 | 2.375 | 2.6 | 10.57 | 45.07 | 47.72 | 7.21 |

FIG. 3C illustrates identifying reservoir fluid and selecting core sample for CO₂ HnP analysis. The HnP analysis core samples are selected based on oil saturation combined with sample porosity. The samples with largest value of the product of oil saturation and porosity are ranked highest for the HnP testing. Examination of oil saturation reported from the GRI tests provides qualitative indication of the residual oil saturation (i.e., oil saturation after primary recovery of hydrocarbons from the reservoir) in the tested formation zones. As shown in FIG. 3C, residual oil saturation values of individual core-plugs from GRI tests (denoted as hollow circles according to the legend (332)) are plotted as a function of the sample depth of the core-plugs. In particular, the residual oil saturation values from clusters (332a, 332b, 332c, 332d, 332e, 332f) range from approximately 67% to 14% along a decreasing trend (332) with respect to the sample depth. The clusters (332a, 332b, 332c, 332d) may correspond to core-plugs from four different wells at different geographical locations. Alternatively, the clusters (332a, 332b, 332c, 332d) may correspond to core-plugs from four different sections of a single well. In comparison to the clusters (332a, 332b, 332c, 332d), the clusters (332e, 332f) correspond to core-plugs from two additional wells that contain less residual oil. This is consistent with the expectations based on prior knowledge of the higher thermal maturity and higher GOR for the produced fluids from the geological locations of these two additional wells. This is different from the other four wells, which are expected to be less mature and may produce more oil. Such comparisons allow identification of formation zones with high residual oil saturation to be selected as targets for recovery by enhanced oil recovery (EOR) methods.

FIG. 3D illustrates measuring sample liquid content using NMR. As noted above, the detailed review of the GRI reports (e.g., as depicted in FIG. 3C above) is followed by initial NMR evaluation (i.e., prior to the HnP analysis) of the fluid-content of the samples. FIG. 3D shows a comparison plot of NMR liquid saturation to GRI reported liquid saturation of the core-plugs in the six clusters (332a, 332b, 332c, 332d, 332e, 332f) according to the legend (340). The comparison demonstrates excellent agreement between the two measurements. The scattering of the data points in the comparison plot along the 1:1 line (341) is because the GRI sample locations are often offset from the core-plug location by a few inches. Specifically, the GRI reports are based on companion samples of the core-plugs for the NMR scan. Although each core-plug and companion sample are taken from the same rock core, the two locations cannot physically overlap and are typically a few inches apart from each other.

FIG. 3E shows a test apparatus used for CO₂ HnP test to extract oil from selected core samples. In one or more embodiments, the test apparatus is part of the core sample analysis system (160) depicted in FIG. 1 above. As shown in FIG. 3E, the test apparatus (350) includes the experiment control and data-logging software display (351a) with pressure gauges (351b) to control the CO₂ charge pressure, multiple pressure cells (352) in which the core-samples are loaded and charged (i.e., pressurized) with CO₂, the condenser (353a) and the separator tube (353b) to cool the effluent gas and collect the condensate, and the wet-test-meter (354) to measure the excess gas. The excess gas measurement is the total amount of gas that was stored in the sample chamber containing the core-plug including the gas in the space outside the core-plug, gas in the pore-space, gas dissolved in the fluids contained in the pore-space and the gas adsorbed in the rock-matrix. Measuring this amount of gas after each cycle allows determination of the “gas-storage capacity” (per mass or volume) of the core-plug and its change with subsequent cycles. This estimate is very important for the determination of CO₂ sequestration capacity of different rock-formations represented by the core-plugs. Core-plugs that are tested are weighed before loading into a pressure vessel to be pressured up with CO₂ or other suitable gas to about 4000 psia and allowed to soak for several hours to a few days. Soaking allows CO₂ to interact with the fluids (e.g., oil) in the core sample and swells the oil where lighter hydrocarbon components vaporize into gas phase surrounding the core-plugs under test. Following soaking, the liquid/gas mixture surrounding the core sample is produced via a separator to measure the condensed liquids and the produced gas. Completion of this step constitutes a single HnP cycle.

FIG. 3F shows the pressure profile during a sequence of HnP cycles in a typical HnP test. In each HnP cycle, the pressure vessel is raised to approximately 4000 psia with a soak time of approximately 100 hours before a draw-down period (360) to atmospheric pressure during which gas and condensate are produced.

FIG. 3G shows examples of fluids (371) collected by the HnP test and the appearance of core samples (372) after the test. In many cases, multiple HnP cycles are performed in EOR of the unconventional reservoir to increase penetration and recovery further from the wellbore. For laboratory testing with additional cycles, the HnP cycle is repeated until the sample weight stabilizes. The produced liquid is analyzed by measuring the amount and density to provide an indication of hydrocarbon quality. Final sample weight and post-testing NMR evaluation allows determination of the hydrocarbons extracted from the sample. The produced liquid is also analyzed using gas-chromatography to determine the hydrocarbon composition. A gas-chromatography plot showing the hydrocarbon composition in the produced liquid from the HnP test is shown in FIG. 3H.

FIG. 3I shows a bar chart illustrating initial fluid contents (381a, 381b, 381c, 381d, 381e) of 5 different core-plugs where the volume fractions of oil, gas, and water are represented as % of pore volume based on reported porosity in GRI test reports. The extracted fluid amounts with increasing number of HnP cycles for these core-plugs are shown as % of pore volume in corresponding data bars (382a, 382b, 382c, 382d, 382e) according to the legend (380). The percentage values shown in each of the data bars (382a, 382b, 382c, 382d, 382e) are also tabulated according to the legend (380) and below the corresponding data bar for HnP cycle 1 followed by cycles 2, 3, and 4. It is observed that the largest oil volume is recovered in the first HnP cycle 1 and smaller volume is further recovered in subsequent cycles 2, 3, and 4.

FIG. 3J shows a bar chart with associated tabulated data of recovered residual oil as percentage of pore volume based on residual oil saturations determined from GRI reports and NMR measurements in the HnP test. The bar chart and tabulated data are for six different core-plugs denoted as A-HZ6, B-HZ6, C-HZ6, 7HB, 7HC, and 2Ha according to the legend (390). Further, the bar chart and tabulated data for these six core-plugs are organized into sections (391, 392, 393, 394) corresponding to measurements obtained after HnP cycle 1 followed by cycles 2, 3, and 4, respectively. It is noted that, in the reservoir, the samples are not subject to atmospheric pressure as in the HnP test equipment, therefore the recoveries from the reservoir may be lower than that measured in the HnP analysis. However, a portion of the difference may be compensated by the reservoir pressure and temperature being higher than the pressure and temperature conditions in the HnP test equipment.

Experimental results discussed above in reference to FIGS. 3A-3J show that a volume of oil, including any extracted or solubilized bitumen, representing up to 30% of the total pore volume could be extracted using the proposed process. This oil is extracted from as-received core samples representing various geological formation zones of interest in extremely depleted reservoir conditions. As measured by NMR in the HnP analysis or reported in GRI reports, the extracted oil is estimated to represent more than 50% of the residual oil, which may correspond to approximately 90% of original oil in place for unconventional reservoirs. Accordingly, the results of oil extraction from core samples representing different geological formation zones in a reservoir are used to rank various formation zones with regards to amount of oil volume that can be extracted by the selected EOR process.

An example dataset is tabulated in TABLE 2 to demonstrate how the results from CO₂ HnP analysis are used to rank formation zones. TABLE 2 shows GRI and HnP analysis results for seven selected samples denoted as A, B, C, D, E, F, and G. It is observed that samples F and G have higher porosity (i.e., ability to store fluids per bulk volume) compared to other samples. Samples D and E have the highest amount of oil per pore volume (59%) compared to sample C which only has 26% of pore volume occupied by oil. The row labeled “after Cycle 3” represents oil that can be recovered after three HnP cycles from each sample (represented as percentage of pore volume available in the sample) and indicates that sample E has the highest recovery of approximately 40% of the original oil in place but has very low porosity. However, the samples G and F with the highest original porosity values has recovered the most oil per bulk reservoir volume, as indicated in the bottom row.

TABLE 2
Summary of Residual and Recovered Oil

| | A | B | C | D | E | F | G |
|---|---|---|---|---|---|---|---|
| Porosity^{a} | 5.3% | 6.7% | 7.8% | 2.7% | 2.7% | 8.8% | 11.9% |
| Gas Saturation^{a} | 40% | 51% | 59% | 32% | 32% | 52.2% | 54.7% |
| Oil Saturation^{a} | 39% | 27% | 26% | 59% | 59% | 42.1% | 40.7% |
| Water Saturation^{a} | 20% | 22% | 15% | 8% | 8% | 5.7% | 4.6% |
| After Cycle 1: | 19.11% | 7.11% | 12.60% | 24.11% | 31.08% | 24.47% | 16.33% |
| After Cycle 2: | 26.18% | 13.34% | 17.04% | | | | 25.30% |
| After Cycle 3: | 31.55% | 17.43% | 19.34% | 31.73% | 40.46% | 31.40% | 30.47% |
| Oil Recovered/ | 1.67% | 1.17% | 1.5% | 0.8% | 1.09% | 2.77% | 3.6% |
| Rock bulk-volume | | | | | | | |

^{a}measured from GRI

FIG. 3K illustrates another example of 33 tested samples ranked from highest to lowest recovery per reservoir bulk volume based on the GRI and HnP analysis results. As shown in FIG. 3K, the reservoirs layers represented by samples 1, 2, and 3 have the highest potential for increasing recovery and EUR based on the GRI and HnP analysis results and are identified as target for EOR production.

FIG. 3L illustrates the increase in gas permeability and CO₂ injectivity and storage capacity of two core-plugs after HnP cycles. As shown in FIG. 3L, the initial liquid saturation (395a) for a core-plug (denoted as 8Hb according to the legend (395)) is 55% with an estimated gas relative permeability of 0.005 mD (from GRI analysis). After three HnP cycles, the oil saturation of the core-plug 8Hb is reduced to 22% and the gas relative permeability increases to 0.045 mD, which is approximately 10 times larger than at the initial conditions. The saturation and permeability values of the other well denoted as 8Ha show a similar trend. Therefore, the CO₂ HnP process has the potential to increase the effective gas permeability and the production nearly 10 times. In other words, this process can significantly increase production from gas wells suffering from productivity loss due to condensate build-up. Reduction in oil saturation after multiple CO₂ HnP cycles is expected to increase the storage capacity if the depleted reservoir is used for CO₂ sequestration. In the oil and gas industry, the term “sequestration” refers to storing CO₂ in subsurface structures such as oil reservoirs, natural gas deposits, un-mineable coal seams, deep saline formations, shale rich in oil or gas, and basalt formations.

The initial CO₂ storage capacity may be represented by gas saturation in the depleted reservoir, which can be represented approximately by the gas saturation in the core samples reported in GRI test reports as Eq. (1) below.

CO₂ storage capacity/Acre-ft of reservoir volume (Ton/acre-ft)

$$= ϕ {S}_{g} {ρ}_{co 2} \times 43560 {ft}^{3} / acre - ft \qquad Eq . ( 1 )$$

In Eq. (1), the density of carbon dioxide of 0.645 gm/cm³ at approximate reservoir temperature (275 degree F.) and a target storage pressure of 5000 psia are used.

The increase in CO₂ storage capacity after CO₂ HnP cycles is calculated by converting the weight of oil extracted to the volume of oil removed using an oil density of 0.8 gm/cm³ in Eq. (2) below.

Increase CO₂ storage capacity/Acre-ft of reservoir volume (Ton/acre-ft)

$$Eq . ( 2 )$$

$$= ( Wt . - change / 0.8 / core bulk - volume ) * {ρ}_{co 2} \times 43560 {ft}^{3} / acre - ft$$

Accordingly, the total CO₂ storage capacity after CO₂ HnP cycles is calculated by adding the results of Eq. (1) and Eq. (2).

FIG. 3M shows a bar chart that compares the available CO₂ storage capacity of 35 samples before and after the CO₂ HnP cycles using 5000 psia pressure at reservoir temperature. The 35 samples are taken from different rock cores of different wells and correspond to various formation zones in an unconventional reservoir. Each bar in the bar chart and corresponding tabulated data collectively show the before and after available CO₂ storage capacity values measured in Tons/acre-ft according to the vertical coordinates and the legend (396). It is noted that additional CO₂ can be stored via “adsorption” in the organic matrix of the unconventional reservoirs. It is important to note that the CO₂ storage capacity is almost doubled by the CO₂ HnP cycles.

Embodiments described above have the following advantages: (i) enabling assessment of reservoir fluid amount based on rock samples to select target formation zones for enhanced oil recovery and/or enhancing gas productivity by condensate-blockage removal, (ii) allowing relative ranking of various geological formations/wells with regards to their potential for additional oil recovery, (iii) increasing gas permeability and productivity of unconventional reservoirs by an order of magnitude, and (iv) increasing the CO₂ injectivity by an order of magnitude and nearly doubling the CO₂ storage capacity after EOR production of the unconventional reservoirs.

Embodiments may be implemented on a computer system. FIG. 4 is a block diagram of a computer system (402) used to provide computational functionalities associated with described algorithms, methods, functions, processes, flows, and procedures as described in the instant disclosure, according to an implementation. The illustrated computer (402) is intended to encompass any computing device such as a high performance computing (HPC) device, a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (402) may include a computer that includes an input device, such as a keypad, keyboard, touch screen, or other device that can accept user information, and an output device that conveys information associated with the operation of the computer (402), including digital data, visual, or audio information (or a combination of information), or a GUI.

The computer (402) can serve in a role as a client, network component, a server, a database or other persistency, or any other component (or a combination of roles) of a computer system for performing the subject matter described in the instant disclosure. The illustrated computer (402) is communicably coupled with a network (430). In some implementations, one or more components of the computer (402) may be configured to operate within environments, including cloud-computing-based, local, global, or other environment (or a combination of environments).

At a high level, the computer (402) is an electronic computing device operable to receive, transmit, process, store, or manage data and information associated with the described subject matter. According to some implementations, the computer (402) may also include or be communicably coupled with an application server, e-mail server, web server, caching server, streaming data server, business intelligence (BI) server, or other server (or a combination of servers).

The computer (402) can receive requests over network (430) from a client application (for example, executing on another computer (402)) and responding to the received requests by processing the said requests in an appropriate software application. In addition, requests may also be sent to the computer (402) from internal users (for example, from a command console or by other appropriate access method), external or third-parties, other automated applications, as well as any other appropriate entities, individuals, systems, or computers.

Each of the components of the computer (402) can communicate using a system bus (403). In some implementations, any or all of the components of the computer (402), both hardware or software (or a combination of hardware and software), may interface with each other or the interface (404) (or a combination of both) over the system bus (403) using an application programming interface (API) (412) or a service layer (413) (or a combination of the API (412) and service layer (413). The API (412) may include specifications for routines, data structures, and object classes. The API (412) may be either computer-language independent or dependent and refer to a complete interface, a single function, or even a set of APIs. The service layer (413) provides software services to the computer (402) or other components (whether or not illustrated) that are communicably coupled to the computer (402). The functionality of the computer (402) may be accessible for all service consumers using this service layer. Software services, such as those provided by the service layer (413), provide reusable, defined business functionalities through a defined interface. For example, the interface may be software written in JAVA, C++, or other suitable language providing data in extensible markup language (XML) format or other suitable format. While illustrated as an integrated component of the computer (402), alternative implementations may illustrate the API (412) or the service layer (413) as stand-alone components in relation to other components of the computer (402) or other components (whether or not illustrated) that are communicably coupled to the computer (402). Moreover, any or all parts of the API (412) or the service layer (413) may be implemented as child or sub-modules of another software module, enterprise application, or hardware module without departing from the scope of this disclosure.

The computer (402) includes an interface (404). Although illustrated as a single interface (404) in FIG. 4, two or more interfaces (404) may be used according to particular needs, desires, or particular implementations of the computer (402). The interface (404) is used by the computer (402) for communicating with other systems in a distributed environment that are connected to the network (430). Generally, the interface (404) includes logic encoded in software or hardware (or a combination of software and hardware) and operable to communicate with the network (430). More specifically, the interface (404) may include software supporting one or more communication protocols associated with communications such that the network (430) or interface's hardware is operable to communicate physical signals within and outside of the illustrated computer (402).

The computer (402) includes at least one computer processor (405).

Although illustrated as a single computer processor (405) in FIG. 4, two or more processors may be used according to particular needs, desires, or particular implementations of the computer (402). Generally, the computer processor (405) executes instructions and manipulates data to perform the operations of the computer (402) and any algorithms, methods, functions, processes, flows, and procedures as described in the instant disclosure.

The computer (402) also includes a memory (406) that holds data for the computer (402) or other components (or a combination of both) that can be connected to the network (430). For example, memory (406) can be a database storing data consistent with this disclosure. Although illustrated as a single memory (406) in FIG. 4, two or more memories may be used according to particular needs, desires, or particular implementations of the computer (402) and the described functionality. While memory (406) is illustrated as an integral component of the computer (402), in alternative implementations, memory (406) can be external to the computer (402).

The application (407) is an algorithmic software engine providing functionality according to particular needs, desires, or particular implementations of the computer (402), particularly with respect to functionality described in this disclosure. For example, the application (407) can serve as one or more components, modules, applications, etc. Further, although illustrated as a single application (407), the application (407) may be implemented as multiple applications (407) on the computer (402). In addition, although illustrated as integral to the computer (402), in alternative implementations, the application (407) can be external to the computer (402).

There may be any number of computers (402) associated with, or external to, a computer system containing computer (402), each computer (402) communicating over network (430). Further, the term “client,” “user,” and other appropriate terminology may be used interchangeably as appropriate without departing from the scope of this disclosure. Moreover, this disclosure contemplates that many users may use one computer (402), or that one user may use multiple computers (402).

In some embodiments, the computer (402) is implemented as part of a cloud computing system. For example, a cloud computing system may include one or more remote servers along with various other cloud components, such as cloud storage units and edge servers. In particular, a cloud computing system may perform one or more computing operations without direct active management by a user device or local computer system. As such, a cloud computing system may have different functions distributed over multiple locations from a central server, which may be performed using one or more Internet connections. More specifically, cloud computing system may operate according to one or more service models, such as infrastructure as a service (IaaS), platform as a service (PaaS), software as a service (SaaS), mobile “backend” as a service (MBaaS), serverless computing, artificial intelligence (AI) as a service (AlaaS), and/or function as a service (FaaS).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A method to perform a field operation of an unconventional reservoir, comprising:
selecting, from a plurality of rock samples and based on test reports of corresponding companion rock samples, a plurality of selected rock samples representing rock characteristics of a plurality of formation zones in the unconventional reservoir;
performing, using a rock sample test apparatus, a sequence of rock sample test cycles on each selected rock sample of the plurality of selected rock samples to generate rock sample test results;
generating, based on the rock sample test results, a ranking of the plurality of selected rock samples representing enhanced oil recovery (EOR) potential of the plurality of formation zones;
selecting, from the plurality of formation zones and based on the ranking of the plurality of selected rock samples, a first target formation zone having a first EOR potential meeting a first pre-determined criterion; and
performing, based at least on the first EOR potential of the first target formation zone, the field production of the unconventional reservoir.

2. The method of claim 1,
wherein the plurality of rock samples are obtained from the plurality of formation zones subsequent to primary depletion of the unconventional reservoir,
wherein the rock sample test apparatus comprises a Huff-and-Puff (HnP) test apparatus,
wherein the sequence of rock sample test cycles comprises a sequence of HnP cycles, and
wherein the field operation comprises a first EOR production operation to extract hydrocarbons from the first target formation zone.

3. The method of claim 2, further comprising:
determining, based on the rock sample test results, a storage capacity for sequestration of the first target formation zone,
wherein the field operation further comprises a sequestration operation to store carbon dioxide (CO₂) gas in the first target formation zone subsequent to the first EOR production operation.

4. The method of claim 3, further comprising:
further selecting, from the plurality of formation zones and based on the ranking of the plurality of selected rock samples, a second target formation zone having a second EOR potential meeting the pre-determined criterion; and
performing, based on the storage capacity for sequestration of the first target formation zone and the second EOR potential of the second target formation zone, a second EOR production operation to extract hydrocarbons from the second target formation zone,
wherein the second EOR production operation comprises an injection operation subsequent to the first EOR production operation and using the CO₂ gas stored in the first target formation zone.

5. The method of claim 1, further comprising:
measuring, prior to the sequence of rock sample test cycles, said each selected rock sample to generate a prior weight and volume measurement;
measuring, subsequent to the sequence of rock sample test cycles, said each selected rock sample to generate a subsequent weight and volume measurement; and
determining, by at least comparing the prior weight and volume measurement and the subsequent weight and volume measurement, a residual oil amount of said each selected rock sample,
wherein the rock sample test results comprise the residual oil amount of said each selected rock sample.

6. The method of claim 5, further comprising:
measuring, prior to the sequence of rock sample test cycles, said each selected rock sample to generate a prior nuclear magnetic resonance (NMR) measurement; and
measuring, subsequent to the sequence of rock sample test cycles, said each selected rock sample to generate a subsequent NMR measurement,
determining the residual oil amount of said each selected rock sample is further based on comparing the prior NMR measurement and the subsequent NMR measurement.

7. The method of claim 1,
wherein the test reports of corresponding companion rock samples comprise Gas Research Institute (GRI) reports.

8. A core sample analysis system to facilitate a field operation of an unconventional reservoir, comprising:
a computer processor; and
memory storing instructions, when executed by the computer processor comprising functionality for:
selecting, from a plurality of rock samples and based on test reports of corresponding companion rock samples, a plurality of selected rock samples representing rock characteristics of a plurality of formation zones in the unconventional reservoir;
performing, using a rock sample test apparatus, a sequence of rock sample test cycles on each selected rock sample of the plurality of selected rock samples to generate rock sample test results;
generating, based on the rock sample test results, a ranking of the plurality of selected rock samples representing enhanced oil recovery (EOR) potential of the plurality of formation zones;
selecting, from the plurality of formation zones and based on the ranking of the plurality of selected rock samples, a first target formation zone having a first EOR potential meeting a first pre-determined criterion; and
facilitating, based at least on the first EOR potential of the first target formation zone, the field production of the unconventional reservoir.

9. The core sample analysis system of claim 8,
wherein the plurality of rock samples are obtained from the plurality of formation zones subsequent to primary depletion of the unconventional reservoir,
wherein the rock sample test apparatus comprises a Huff-and-Puff (HnP) test apparatus,
wherein the sequence of rock sample test cycles comprises a sequence of HnP cycles, and
wherein the field operation comprises a first EOR production operation to extract hydrocarbons from the first target formation zone.

10. The core sample analysis system of claim 9, the instructions, when executed by the computer processor further comprising functionality for:
determining, based on the rock sample test results, a storage capacity for sequestration of the first target formation zone,
wherein the field operation further comprises a sequestration operation to store carbon dioxide (CO₂) gas in the first target formation zone subsequent to the first EOR production operation.

11. The core sample analysis system of claim 10, the instructions, when executed by the computer processor further comprising functionality for:
further selecting, from the plurality of formation zones and based on the ranking of the plurality of selected rock samples, a second target formation zone having a second EOR potential meeting the pre-determined criterion; and
facilitating, based on the storage capacity for sequestration of the first target formation zone and the second EOR potential of the second target formation zone, a second EOR production operation to extract hydrocarbons from the second target formation zone,
wherein the second EOR production operation comprises an injection operation subsequent to the first EOR production operation and using the CO₂ gas stored in the first target formation zone.

12. The core sample analysis system of claim 8, the instructions, when executed by the computer processor further comprising functionality for:
measuring, prior to the sequence of rock sample test cycles, said each selected rock sample to generate a prior weight and volume measurement;
measuring, subsequent to the sequence of rock sample test cycles, said each selected rock sample to generate a subsequent weight and volume measurement; and
determining, by at least comparing the prior weight and volume measurement and the subsequent weight and volume measurement, a residual oil amount of said each selected rock sample,
wherein the rock sample test results comprise the residual oil amount of said each selected rock sample.

13. The core sample analysis system of claim 12, the instructions, when executed by the computer processor further comprising functionality for:
measuring, prior to the sequence of rock sample test cycles, said each selected rock sample to generate a prior nuclear magnetic resonance (NMR) measurement; and
measuring, subsequent to the sequence of rock sample test cycles, said each selected rock sample to generate a subsequent NMR measurement,
determining the residual oil amount of said each selected rock sample is further based on comparing the prior NMR measurement and the subsequent NMR measurement.

14. The core sample analysis system of claim 8,
wherein the test reports of corresponding companion rock samples comprise Gas Research Institute (GRI) reports.

15. A system comprising:
a well control system for performing a field operation of an unconventional reservoir; and
a core sample analysis system comprising a computer processor and memory storing instructions, when executed by the computer processor comprising functionality for:
selecting, from a plurality of rock samples and based on test reports of corresponding companion rock samples, a plurality of selected rock samples representing rock characteristics of a plurality of formation zones in the unconventional reservoir;
performing, using a rock sample test apparatus, a sequence of rock sample test cycles on each selected rock sample of the plurality of selected rock samples to generate rock sample test results;
generating, based on the rock sample test results, a ranking of the plurality of selected rock samples representing enhanced oil recovery (EOR) potential of the plurality of formation zones;
selecting, from the plurality of formation zones and based on the ranking of the plurality of selected rock samples, a first target formation zone having a first EOR potential meeting a first pre-determined criterion; and
facilitating, based at least on the first EOR potential of the first target formation zone, the well control system to perform the field production of the unconventional reservoir.

16. The system of claim 15,
wherein the plurality of rock samples are obtained from the plurality of formation zones subsequent to primary depletion of the unconventional reservoir,
wherein the rock sample test apparatus comprises a Huff-and-Puff (HnP) test apparatus,
wherein the sequence of rock sample test cycles comprises a sequence of HnP cycles, and
wherein the field operation comprises a first EOR production operation to extract hydrocarbons from the first target formation zone.

17. The system of claim 16, the instructions, when executed by the computer processor further comprising functionality for:
determining, based on the rock sample test results, a storage capacity for sequestration of the first target formation zone,
wherein the field operation further comprises a sequestration operation to store carbon dioxide (CO₂) gas in the first target formation zone subsequent to the first EOR production operation.

18. The system of claim 17, the instructions, when executed by the computer processor further comprising functionality for:
further selecting, from the plurality of formation zones and based on the ranking of the plurality of selected rock samples, a second target formation zone having a second EOR potential meeting the pre-determined criterion; and
facilitating, based on the storage capacity for sequestration of the first target formation zone and the second EOR potential of the second target formation zone, a second EOR production operation to extract hydrocarbons from the second target formation zone,
wherein the second EOR production operation comprises an injection operation subsequent to the first EOR production operation and using the CO₂ gas stored in the first target formation zone.

19. The system of claim 15, the instructions, when executed by the computer processor further comprising functionality for:
measuring, prior to the sequence of rock sample test cycles, said each selected rock sample to generate a prior weight and volume measurement;
measuring, subsequent to the sequence of rock sample test cycles, said each selected rock sample to generate a subsequent weight and volume measurement; and
determining, by at least comparing the prior weight and volume measurement and the subsequent weight and volume measurement, a residual oil amount of said each selected rock sample,
wherein the rock sample test results comprise the residual oil amount of said each selected rock sample.

20. The system of claim 19, the instructions, when executed by the computer processor further comprising functionality for:
measuring, prior to the sequence of rock sample test cycles, said each selected rock sample to generate a prior nuclear magnetic resonance (NMR) measurement; and
measuring, subsequent to the sequence of rock sample test cycles, said each selected rock sample to generate a subsequent NMR measurement,
determining the residual oil amount of said each selected rock sample is further based on comparing the prior NMR measurement and the subsequent NMR measurement.
